(19) United States
(12) Patent Application Publication
(10) Pub. No.: US 2026/0340562 A1
(43) Pub. Date: Oct. 29, 2026

(54) MODULAR QUALITY MANAGEMENT SYSTEMS AND METHODS

(71) Applicants: Roche Molecular Systems, Inc., Pleasanton, CA (US); Roche Sequencing Solutions, Inc., Pleasanton, CA (US); Roche Diagnostics Operations, Inc., Indianapolis, IN (US); Roche Diagnostics International AG, Rotkreuz (CH); Roche Diagnostics GMBH, Mannheim (DE); Ventana Medical Systems, Inc., Tucson, AZ (US)

(72) Inventors: Meetu ARYA, San Ramon, CA (US); Timothy Patrick BLAIR, Oldwick, NJ (US); Devon Margaux BUREK, Pipersville, PA (US); Adam Robert CLARK, Zionsville, IN (US); Gabriele NEBL, Forst (DE); Amy Marie NELSON, Pendleton, IN (US); Kareena PARRIS, Tucson, AZ (US); Matthias PILZ, Lucerne (CH)

(21) Appl. No.: 19/701,552

(22) Filed: Jun. 8, 2026

Related U.S. Application Data
(63) Continuation of application No. PCT/US2024/060073, filed on Dec. 13, 2024.
(60) Provisional application No. 63/611,651, filed on Dec. 18, 2023.

(51) Int. Cl. G06Q 10/0639; G16H 40/40

(52) U.S. Cl. CPC G06Q 10/06395; G16H 40/40

(57) ABSTRACT

A system for quality control of a medical device or software product including a plurality of quality control (QC) modules, each QC module configured for one or more QC task(s) of a QC process and configured for access by a plurality of users assigned roles and responsibilities for the respective QC task(s), the quality control library configured to store a plurality of QC records including information associated with the QC task(s) and governmental rule(s) or regulation(s) applicable to the respective QC module and medical device product.

### CROSS REFERENCES TO RELATED APPLICATIONS

The present application is a bypass continuation of International Application No. PCT/US 2024/060073 filed Dec. 13, 2024, which claims benefit of priority to U.S. Provisional Application No. 63/611,651, filed Dec. 18, 2023, the entire contents of each of which are herein incorporated by reference.

### BACKGROUND

Numerous elements of the design, testing, manufacture, and the distribution of medical devices rely on effective quality control processes. A medical device must offer safe and effective treatment and/or testing of patients and comply with numerous regulatory requirements. The many aspects of design through distribution and post-market activity must each comport with high quality standards in order to meet these requirements. Manufacturers of these products often run into delays, interruptions, and/or failures in meeting quality control requirements because of the complexity and lack of coordination and/or planning required by the quality control process, particularly for devices with numerous complex software and hardware components.

Controlling the quality of many of these aspects would benefit from coordination and overlap of quality control processes. Improved methods and systems of quality control are thus needed for the effective, safe, and efficient design, manufacture, and distribution of medical devices.

### BRIEF SUMMARY

A modular quality management system includes a library of quality system modules and processes for performing quality control of medical diagnostic and therapy delivery provided by medical devices including instruments and/or software. The system is configured for users to manage module administration, library documents, and tasks including adding modules, assigning users to roles within the modules and responsibility for quality system tasks associated with the modules.

An modular quality management system includes multiple modules each for maintaining and controlling a distinct and respective library module of records pertaining to diagnostic and therapy delivery. In some embodiments, the library includes a “clinical evidence and studies” module for QC tasks pertaining to assessing the quality of clinical evidence and studies of testing or use of the medical device. The module may store governmental rules and regulations pertaining to regulatory approval of the device (e.g., FDA regulations) as well as records of clinical testing and studies utilized in connection with the approval process. One or more leaders and implementers of the module may be assigned for carrying out QC management and implementation of obtaining, storing, and assessing the quality of the device testing and clinical study materials. Enablers may be assigned to perform data analytics of the clinical study and testing data.

Further modules may include a management responsibility module configured for QC tasks that assign users to management roles and responsibilities within a management system module; a management system module configured for QC tasks that train users and assign tasks for management of a quality control process; an improvement and resolution management module configured for QC tasks that track and improve the performance of the QC tasks of the management system module; a post-market module configured for QC tasks that track issues associated with the medical device product after release of the product to users of the product; an information documentation module configured for QC tasks that receive and store information associated with development and distribution of the medical device product; and a training module configured for QC tasks that train users assigned to roles within the quality system modules.

Modules may include an auditing module configured for QC tasks that audit quality system tasks and records of the plurality of modules; a handling, storage, and distribution module configured for QC tasks that assess process(es) of handling, storing, and distribution of the medical device product; a qualification and validation module configured for QC tasks that qualify and validate the medical device product according to specification(s) and/or regulation(s).

Modules may include a purchasing controls module configured for QC tasks that track and assess the requisition of components of the medical device; a service and installation module configured for QC tasks that assess process(es) of servicing and installing the medical device for a user of the device and ensuring a globally aligned service and installation process; a change controls module configured for QC tasks that track and assess changes in the design, manufacture, distribution, installation, and/or servicing of the medical device. Version and change information may be tracked in relation to changes and applicability to particular specifications and/or regulatory rules or regulations. A clinical evidence and studies module is configured for QC tasks that track and assess the quality of clinical evidence and studies of testing or use of the medical device.

After a modular quality management process is initiated for a product, modules may be generated in waves to improve the efficiency and effectiveness of the system. In some embodiments, modules associated with system management, training, and document control are generated in a first wave. Modules associated with auditing, validation, change controls, and purchasing controls are generated in a second wave. Modules associated with design and development, infrastructure, commercialization, regulatory, and local requirements may be generated in a third wave.

A computer-implemented user interface is configured for managing the library of QC modules and implementing a quality system process. The interface is configured for users to manage module administration, library documents, and tasks including adding modules, assigning users to roles within the modules and responsibility for quality system tasks associated with the modules.

In some embodiments, module records pertaining to quality system tasks (e.g., procedures or processes) are structured with fields according to a template that provides graduated levels of detail relating to the tasks. Managing users or implementers are assigned for performing particular tasks or roles. In some embodiments, assignment and/or reminders of a task may be triggered or dynamically generated based on particular timelines and/or the creation and/or (partial) completion of other tasks.

### BRIEF DESCRIPTION OF THE DRAWINGS

The detailed description is set forth with reference to the accompanying figures.

FIG. 1A is a diagram illustrating waves of modules generated in a modular quality management system (mQMS) according to some embodiments.

FIG. 1B is a diagram illustrating a structure of a mQMS library of records according to some embodiments.

FIG. 2 is an illustrative diagram of a user interface for operating a modular quality management system according to some embodiments.

FIG. 3 is an illustrative flow diagram for implementing a modular quality management system process according to some embodiments.

FIG. 4 is an illustrative template interface for representing and displaying information about a quality management process or task in a modular quality management system according to some embodiments.

FIG. 5 is an exemplary representation of a library record in a modular quality management system according to some embodiments.

FIG. 6 illustrates a computer system in a modular quality management system according to some embodiments.

### DETAILED DESCRIPTION

Systems and computer-implemented processes for controlling and improving the quality of medical device products are described. In some embodiments, a modular quality management system is configured with multiple modules for executing quality system tasks such as for the design, manufacture, and distribution of medical device products and software. Each module is configured for managing tasks associated with quality system processes that can include assisting in following regulatory rules or laws, testing of products for meeting particular requirement specifications, assuring consistency and quality of the components, training and use of the products, and minimizing risks in development and production, among others. Each module may be associated with users of the system including implementers or managers of the tasks, coordination between modules, and/or oversight/management of the tasks and/or other users. In some embodiments, enablers of a module may be tasked with performing data analytics and/or risk governance for enabling completion of the tasks (e.g., assessing clinical study data/performance).

The system may include a database of records including information pertaining to the module(s), tasks, and users. A user interface is configured to allow users to access, view, and change the information/records, including adding or removing modules, assigning users to roles/tasks for a module, and adding/modifying information associated with the quality management tasks. In some embodiments, users of the system may be assigned with and notified of tasks, which may be triggered in the system based on the status of other tasks. Information stored in the records may include national or local regulations, design documents, specifications, component sourcing documentation, and testing protocols and results, for example.

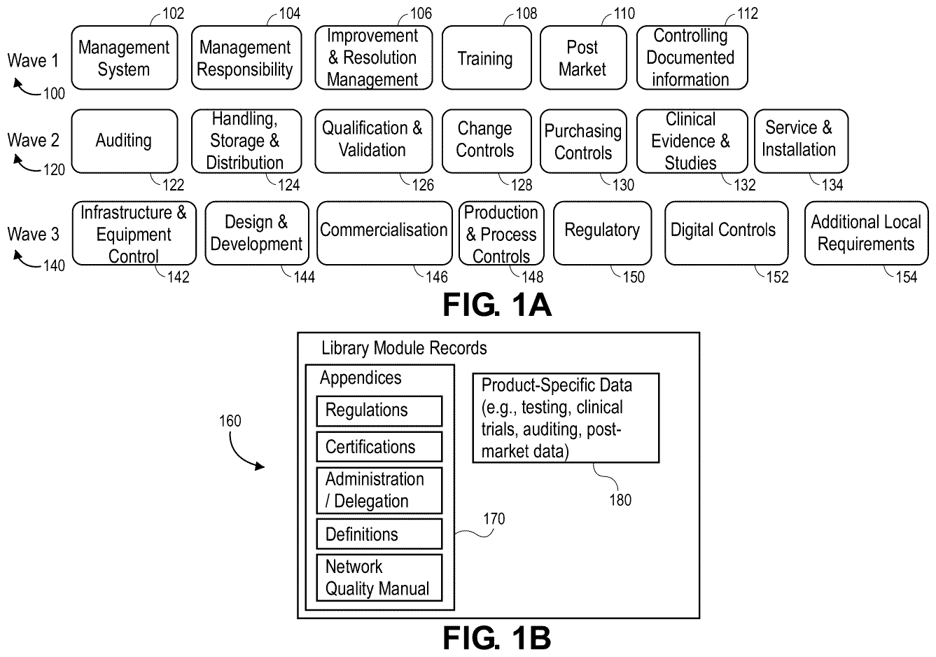

FIG. 1A is a diagram illustrating waves of modules generated in a modular quality management system according to some embodiments. A modular quality management system causes select waves of system modules to be generated and initially configured in the order of the waves during a quality management process. As described further herein, each module may be configured for managing particular quality management tasks. Use of the waves and ordering of the creation of the modules helps optimize the overall efficiency and performance of a quality management system. A first wave 100 is generally directed to modules for management, training, and documentation roles and tasks, a second wave for establishing modules for performing the core quality management tasks including auditing, qualification and validation, purchasing and change controls, and clinical studies, among others. A third wave of modules are configured for managing tasks and documentation particular to the product under development including infrastructure, design, and regulatory requirements, among others.

In a first wave 100, modules for establishing management tasks and roles include a management system module 102 configured for establishing an overriding management structure for implementing quality control over a medical device/software product. The structure may include the modules that will be generated in the system and a delegation of general tasks and roles for quality system management for these modules. A management responsibility module 104 is configured to assign management responsibility roles and tasks in the system including under other modules. An improvement and resolution management module 106 is configured to assign management responsibility roles and tasks for improving management operations. A training module 108 is configured for managing training operations pertaining to use of the quality control management system and product and performing tasks in the quality control management system.

A post market module 110 is configured for assigning roles and tasks for monitoring and improving quality of products after commercialization or release and during use with patients. A module 112 for controlling documented information is configured to manage the creation, storage, and security of documents or records stored that pertain to the quality management process. Such documentation may include design specifications, test protocols, test results, regulatory rules, and other records to be stored within a quality management system library as further described herein.

A second wave 120 of modules, directed to certain principal quality management processes, includes module 122 configured for auditing the development, manufacture, and distribution processes of a product to ensure compliance with quality controls. A handling and storage module 124 is configured for ensuring quality in the handling, storage, and distribution of products. A qualification and validation module 126 is configured for managing the processes of qualifying and validating products, a change controls module 128 is configured for qualifying and tracking changes or proposed changes made in the design, manufacture, and/or distribution of a product. A purchasing controls module 130 is configured for qualifying and tracing the purchasing of product components. A clinical evidence and studies module 132 is configured for qualifying and tracking clinical testing of products. A service and installation module 134 is configured for qualifying and controlling the installation and servicing processes for a product.

A third wave 140 of modules is directed to particular principal processes in the design, manufacture, testing, and distribution of products. The third wave 140 includes an infrastructure and equipment control module 142 configured for qualifying and managing the infrastructure (e.g., factory) and manufacturing equipment used for manufacturing a product. A design and development module 144 configured for qualifying and managing the product design and development process and documentation. In some embodiments, the third wave of modules is generated in connection with establishing and initiating quality system tasks (e.g., management/auditing) and retaining/generating documentation (e.g., applicable regulations, market studies) that can be used for purposes of ensuring quality and compliance in the designing, manufacturing, and marketing/distribution of a product.

A commercialization module 146 is configured for qualifying and managing the commercialization processes for a product (e.g., marketing/advertising). A production and process controls module 148 is configured for qualifying and managing the procedures for production of a product. A regulatory module 150 is configured for qualifying and managing the regulatory process for approval/certification of the product under government regulations and a digital controls module 152 is configured for qualifying and managing digital content associated with the product tracking of digital components of the medical device including, for example, compliance with regulations governing personal identifiable information and data security. An additional local requirements module 154 is configured for qualifying and managing regulatory compliance at a local or regional level (e.g., state and town).

FIG. 1B is a diagram illustrating a structure of a modular quality management system library of records according to some embodiments. A library 160 of records may include records 180 of the specification(s) and design(s) for a product, testing documentation, auditing, and post-market data, for example, wherein copies and version information of these records are stored/managed in an electronic database and storage platform (e.g., cloud). Appendices 170 of quality-related records are also stored within the library and may include, for example, applicable regulations (e.g., FDA), certifications (e.g., FDA approval), a definitions appendix defining terms particular to the product and its development/production/testing, administrative records (e.g., managers/implementers/enablers and associated responsibilities and modules).

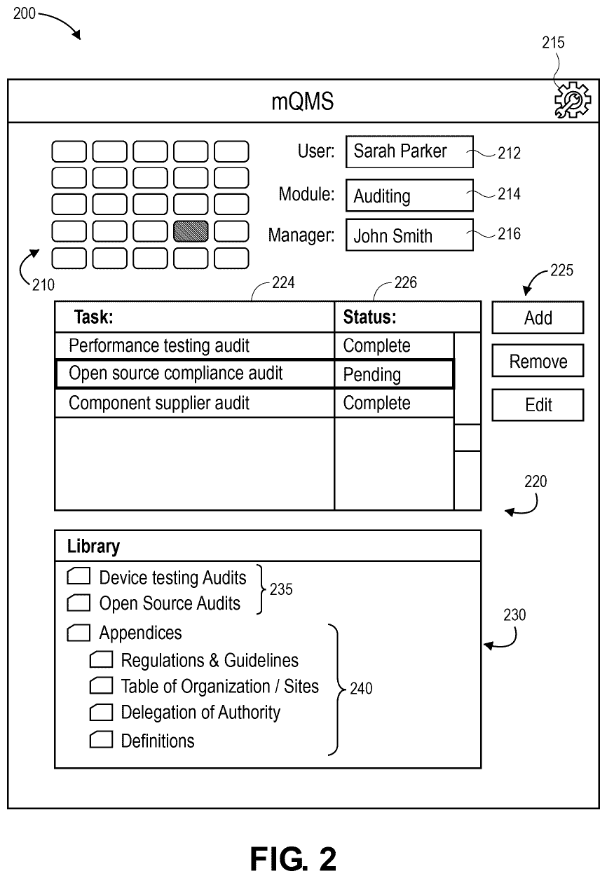

Library 160 may further include a network quality manual detailing the process for creating/configuring a modular quality management system for a medical device/software product (e.g., creating waves of modules, configuring modules, assigning tasks, triggers for tasks). The manual may also include the structures of established modular quality management systems for a particular product, including its respective modules and information about each module and applicable tasks and users/roles. In some embodiments, a graphical user interface permits computer access and management to the library and records stored therein (e.g., as illustrated in FIG. 2).

FIG. 2 is an illustrative diagram of a user interface 200 for operating a modular quality management system according to some embodiments. User interface 200 is configured to allow a user to select among a plurality of modules 210 (e.g., as described in reference to FIG. 1A), for which the current user is identified at 212, the currently selected module identified at 214, and the appointed manager-user identified at 216. A selectable table 220 displays quality management tasks 224 associated with the module and/or active user, and a status 226 associated with each of the tasks. A selectable library menu 230 is also displayed and provided for navigating, viewing, and adding/removing records within the modular quality management library.

Tasks associated with a module may include, for example, audits to be completed under the module where a status may indicate, for example, whether the audit has been completed or what stage the task is at. Selecting a task may prompt a display of further information about the task such as, for example, identifying users assigned to completing or managing the tasks and their progress, documentation/records associated with the task (e.g., audit records), and deadlines for completing a task. For example, selecting a task may cause the library menu 230 to display or highlight library records (e.g., a requirements specification document, regulatory provision) associated with the currently selected task.

A navigable library records interface 230 provides a folder/file view of records/documents within the modular quality management system. The interface provides a user access to records in the library, including the ability to view, add, remove, and modify records at 225. As described further herein, these records/documents can include audit records 235, design, testing, and manufacturing records, appendices 240 including regulations, guidelines, organizational charts, user roles and assignments, definitions, and quality management system manuals.

The records made accessible and shown may be provided in response to and associated with the module selected from modules 210 and/or based on security privileges assigned to a user/account authenticated by the quality management system. Tasks can be assigned to particular users (e.g., implementers/enablers). The tasks can be configured with notifications to alert users of needed actions on a task and with triggers for creating or assigning tasks in response to certain activity in the system (e.g., completion of a task, failure to complete a task on time or to meet a particular quality requirement in the execution of a task).

A configuration tool can be accessed by selecting input 215, from which a module can be further configured. Additional configuration changes may include configuring notifications to users regarding open tasks, and configuring task triggers. Access to certain configuration features may be based on a user/account security authentication. Users may have privileged modular and/or inter-modular access and responsibility over particular module(s) in which they are assigned implementation or management responsibility of respective task(s) within a module.

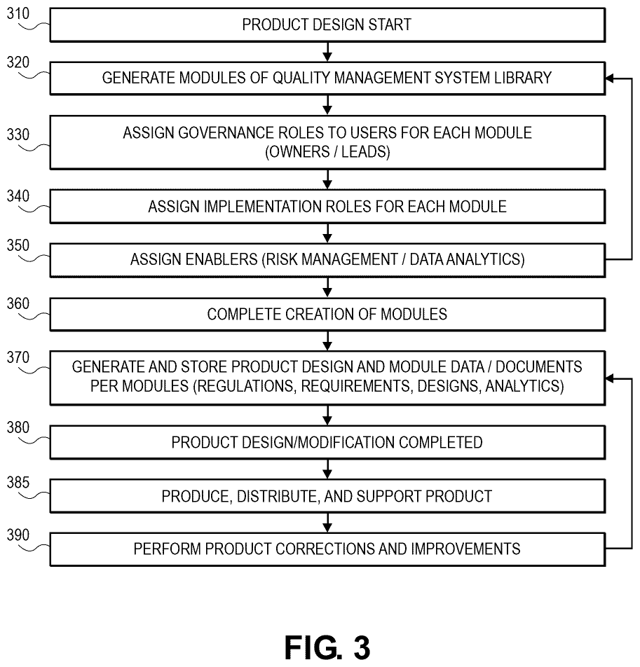

FIG. 3 is an illustrative flow diagram for implementing a modular quality management system process according to some embodiments. At 310, the process for developing a medical device/software product has started. At 320, a modular quality management system is utilized to create quality management modules for the product such as further described herein. In some embodiments, the modules are created in waves such as in reference to FIG. 1A. At 330, creating a module includes assigning users/roles/tasks to the module including, for example, owners/leads or managers assigned tasks for taking management roles for the module and system. For example, a management system module includes tasks for structuring the quality management system for the product, including a general process and schedule for the system and determining the additional modules that should be included for quality management of the product.

At 340, implementation roles (or implementers) for modules are assigned. Implementers may be assigned such tasks as obtaining and reviewing documents as a function of an audit, for example. At 350, enablers are assigned to modules. Enablers may be assigned tasks including risk assessment and data analytics for one or more modules including assessing consistency of meeting/maintaining product design or production requirements associated with the respective module. An enabler may be tasked with reviewing clinical trial data to determine the quality of the data and relate the efficacy and/or risks associated with use of the product based on the data. A cycle of creating modules from block 320 continues until all or a wave of modules is created at 360.

At 370, documents/records used for quality management modules and tasks are obtained. These documents/records may include product design production documentation, testing documentation, and copies of or links to applicable regulatory provisions, for example.

At 380, quality management tasks associated with the design, modification, and/or testing of a product are performed. These tasks may have been assigned in the quality management system and module to particular users (e.g., to implementers, enablers) at block 340. In some embodiments, the system is configured to monitor the status (e.g., completion, quality incident/failure) of these tasks and store information about the status in the system's module library records. Tasks may be repeated and new records created accordingly at 360 as the design, production, testing, and distribution develops or is modified.

In some embodiments, the identification of a task as having been completed may trigger the assignment of other module tasks or their status as needing completion. For example, upon the completion of testing a software product in a qualification and validation module, the system may assign or trigger an open source audit for an implementer to complete or direct under an auditing module. The task may subsequently be identified in a user interface (e.g., interface 200 of FIG. 2) to a user assigned to the task.

At 385, quality management tasks associated with production, distribution, and support of product use are performed. Similar to the tasks performed at block 380, these tasks may be assigned (and/or triggered) in the quality management system and module to particular users. Such tasks may be assigned within a handling, storage, and distribution module or a service and installation module, for example. At 390, in view of quality management operations by the system, corrections and improvements to a product may be performed if needed. Documentation for these improvements/corrections are obtained at 370, and the process of quality management may continue until the production and/or use of a product ceases.

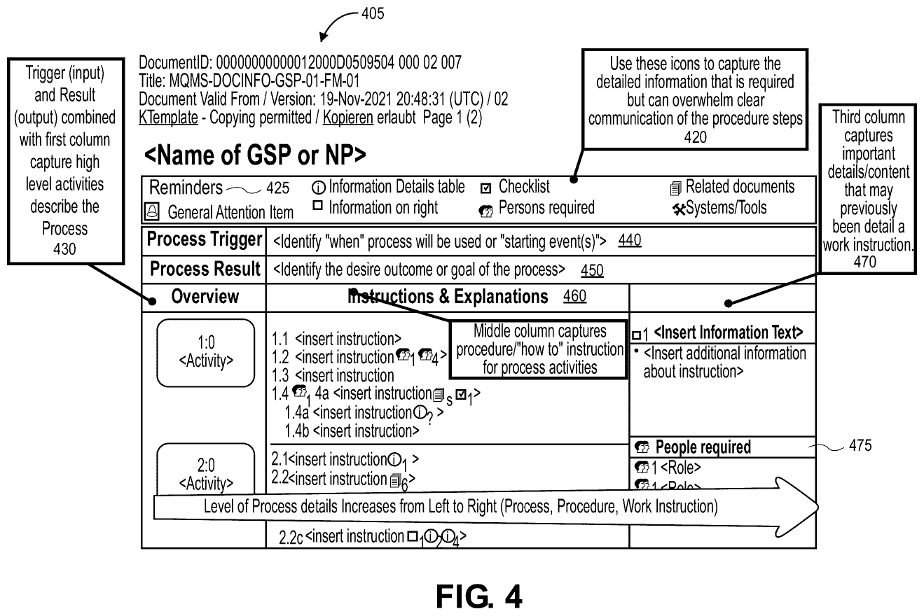

FIG. 4 is an illustrative template interface for representing and displaying information for a quality management process or task in a modular quality management system according to some embodiments. A template 400 provides a framework for storing/displaying information or a document representing a task or process within a modular quality management system. A detailed view selection 420 is used to select what information is to be provided in the interface. A document information field 405 can provide the document filename, version information, document title, and other information about the document. A reminder field 425 displays reminders with respect to the process such as, for example, a due date when module tasks should be completed.

In some embodiments, information displayed in the document is organized so that the level of detail increases from left to right. An overview column 430 provides a general type or name of the task or a process activity. In an instructions and explanations column 460, further detail is provided about the corresponding task/process activity. Column 460 may include explicit instructions for a user (e.g., implementer/enabler) to perform a quality management task or activity such as how to perform an audit or data analysis. An additional information cell 470 provides additional information about the quality management process such as the expected time for completing the process and current progress/status. A people required cell 475 provides a list of the users assigned for completing the process/tasks/activities.

A process trigger field 440 identifies what other activity (e.g., completion of a task/process) triggers the present process or task. A process result field 450 identifies the goal or desired result for completing the process/task and may identify what additional tasks/processes are triggered by the completion of the current process/task. In some embodiments, the interface allows certain users (e.g., managers/administrators) to edit the process document including modifying process triggers 440, expected results 450, instructions 460, additional information 470, and/or assigned users at 475.

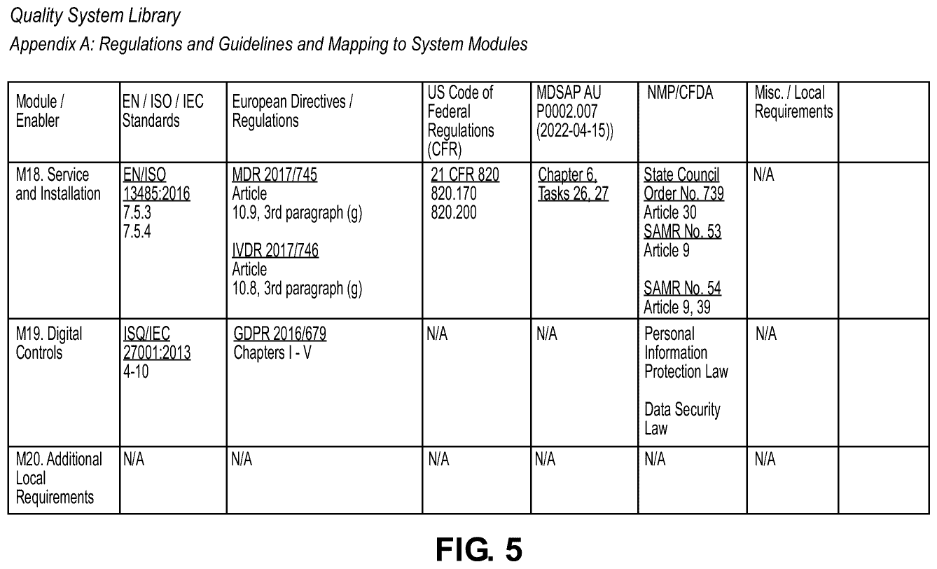

FIG. 5 is an exemplary representation of library record(s) in a modular quality management system according to some embodiments. The record(s) represents particular government regulations pertaining to respective modules of the quality management system (e.g., modules 134, 152, and 154 of FIG. 4). Associated quality management system tasks may include a review by a respective implementer/enabler to obtain and/or review documentation for confirming compliance with the respective regulations (e.g., for digital controls, installation of the devices).

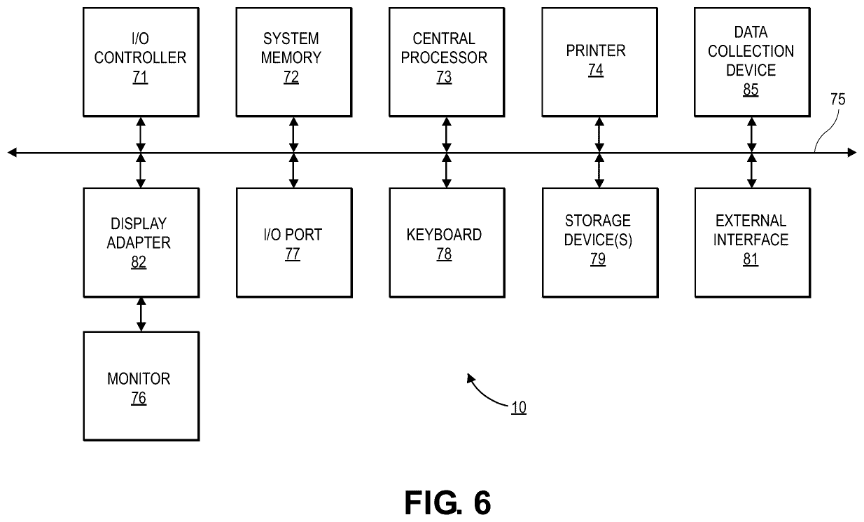

Any of the computer systems mentioned herein, such as for hosting the systems and implementing the processes described for managing liver disease, may utilize any suitable number of subsystems. Examples of such subsystems are shown in FIG. 6 in computer system 10. In some embodiments, a computer system includes a single computer apparatus, where the subsystems can be the components of the computer apparatus. In other embodiments, a computer system can include multiple computer apparatuses, each being a subsystem, with internal components. A computer system can include desktop and laptop computers, tablets, mobile phones and other mobile devices. In some embodiments, a cloud infrastructure (e.g., Amazon Web Services), a graphical processing unit (GPU), etc., can be used to implement the disclosed techniques.

The subsystems shown in FIG. 6 are interconnected via a system bus 75. Additional subsystems such as a printer 74, keyboard 78, storage device(s) 79, monitor 76, which is coupled to display adapter 82, and others are shown. Peripherals and input/output (I/O) devices, which couple to I/O controller 71, can be connected to the computer system by any number of means known in the art such as input/output (I/O) port 77 (e.g., USB, FireWire®). For example, I/O port 77 or external interface 81 (e.g. Ethernet, Wi-Fi, etc.) can be used to connect computer system 10 to a wide area network such as the Internet, a mouse input device, or a scanner. The interconnection via system bus 75 allows the central processor 73 to communicate with each subsystem and to control the execution of a plurality of instructions from system memory 72 or the storage device(s) 79 (e.g., a fixed disk, such as a hard drive, or optical disk), as well as the exchange of information between subsystems. The system memory 72 and/or the storage device(s) 79 may embody a computer readable medium. Another subsystem is a data collection device 85, such as a camera, microphone, accelerometer, and the like. Any of the data mentioned herein can be output from one component to another component and can be output to the user.

A computer system can include a plurality of the same components or subsystems, e.g., connected together by external interface 81 or by an internal interface. In some embodiments, computer systems, subsystem, or apparatuses can communicate over a network. In such instances, one computer can be considered a client and another computer a server, where each can be part of a same computer system. A client and a server can each include multiple systems, subsystems, or components.

Aspects of embodiments can be implemented in the form of control logic using hardware (e.g. an application specific integrated circuit or field programmable gate array) and/or using computer software with a generally programmable processor in a modular or integrated manner. As used herein, a processor includes a single-core processor, multi-core processor on a same integrated chip, or multiple processing units on a single circuit board or networked. Based on the disclosure and teachings provided herein, a person of ordinary skill in the art will know and appreciate other ways and/or methods to implement embodiments of the present invention using hardware and a combination of hardware and software.

Any of the software components or functions described in this application may be implemented as software code to be executed by a processor using any suitable computer language such as, for example, Java, C, C++, C #, Objective-C, Swift, or scripting language such as Perl or Python using, for example, conventional or object-oriented techniques. The software code may be stored as a series of instructions or commands on a computer readable medium for storage and/or transmission. A suitable non-transitory computer readable medium can include random access memory (RAM), a read only memory (ROM), a magnetic medium such as a hard-drive or a floppy disk, or an optical medium such as a compact disk (CD) or DVD (digital versatile disk), flash memory, and the like. The computer readable medium may be any combination of such storage or transmission devices.

Such programs may also be encoded and transmitted using carrier signals adapted for transmission via wired, optical, and/or wireless networks conforming to a variety of protocols, including the Internet. As such, a computer readable medium may be created using a data signal encoded with such programs. Computer readable media encoded with the program code may be packaged with a compatible device or provided separately from other devices (e.g., via Internet download). Any such computer readable medium may reside on or within a single computer product (e.g. a hard drive, a CD, or an entire computer system), and may be present on or within different computer products within a system or network. A computer system may include a monitor, printer, or other suitable display for providing any of the results mentioned herein to a user.

Any of the methods described herein may be totally or partially performed with a computer system including one or more processors, which can be configured to perform the steps. Thus, embodiments can be directed to computer systems configured to perform the steps of any of the methods described herein, potentially with different components performing a respective steps or a respective group of steps. Although presented as numbered steps, steps of methods herein can be performed at a same time or in a different order. Additionally, portions of these steps may be used with portions of other steps from other methods. Also, all or portions of a step may be optional. Additionally, any of the steps of any of the methods can be performed with modules, units, circuits, or other means for performing these steps.

The specific details of particular embodiments may be combined in any suitable manner without departing from the spirit and scope of embodiments of the invention. However, other embodiments of the invention may be directed to specific embodiments relating to each individual aspect, or specific combinations of these individual aspects.

The above description of example embodiments of the invention has been presented for the purposes of illustration and description. It is not intended to be exhaustive or to limit the invention to the precise form described, and many modifications and variations are possible in light of the teaching above.

A recitation of “a”, “an” or “the” is intended to mean “one or more” unless specifically indicated to the contrary. The use of “or” is intended to mean an “inclusive or,” and not an “exclusive or” unless specifically indicated to the contrary. Reference to a “first” component does not necessarily require that a second component be provided. Moreover reference to a “first” or a “second” component does not limit the referenced component to a particular location unless expressly stated.

All patents, patent applications, publications, and descriptions mentioned herein are incorporated by reference in their entirety for all purposes. None is admitted to be prior art.

We claim:

1. A system for quality control of a medical device or software product, the system comprising:
a) an electronic quality control library comprising a plurality of quality control (QC) modules, each QC module configured for one or more QC task(s) of a QC process and configured for access by a plurality of users assigned roles and responsibilities for the respective QC task(s), the quality control library configured to store a plurality of QC records including information associated with the QC task(s) and governmental rule(s) or regulation(s) applicable to the respective QC module and medical device product;
b) one or more processors programmed and configured to:
c) provide a user interface configured for managing the library of QC modules and implementing a quality control process, wherein the managing and implementing includes adding or removing modules, assigning users to roles within the modules and responsibility for quality control tasks associated with the modules, and adding, removing, or modifying the QC records of a respective module.

2. The system of claim 1 wherein the plurality of quality control modules comprises two or more of:
a) a management responsibility module configured for QC tasks that assign users to management roles and responsibilities within a management system module;
b) a management system module configured for QC tasks that train users and assign tasks for management of a quality control process;
c) an improvement and resolution management module configured for QC tasks that track and to improve performance of the QC tasks of the management system module;
d) a post-market module configured for QC tasks that track issues associated with the medical device product after release of the product to users of the product;
e) an information documentation module configured for QC tasks that obtain and store information associated with development and distribution of the medical device product; and
f) a training module configured for QC tasks that train users assigned to roles within the quality control modules.

3. The system of claim 1 wherein the plurality of quality control modules comprises two or more of:
a) an auditing module configured for QC tasks that audit quality control tasks and records of the plurality of modules;
b) a handling, storage, and distribution module configured for QC tasks that assess process(es) of handling, storing, and distribution of the medical product;
c) a qualification and validation module configured for QC tasks that qualify and validate the medical device product according to specification(s) and/or regulation(s);
d) a purchasing controls module configured for QC tasks that track and assess requisition of components of the medical device;
e) a service and installation module configured for QC tasks that assess process(es) of servicing and installing the medical device for a user of the device;
f) a change controls module configured for QC tasks that track and assess changes in designs, manufacture, installation, and/or servicing of the medical device;
g) a clinical evidence and studies module configured for QC tasks that track and assess quality of clinical evidence and studies of testing or use of the medical device.

4. The system of claim 1, wherein the one or more processors and the user interface are further configured for:
assigning and managing governance and implementation roles for users of the interface and library, wherein the governance roles are assigned privileged modular and/or inter-modular access and responsibility over respective module(s) and wherein implementation roles are assigned modular access and implementation responsibility of respective task(s) within a module.

5. The system of claim 1, wherein the one or more processors and the user interface are further configured for:
assigning and managing enabler roles for each of the modules, wherein the enabler roles are assigned tasks for data analytics and risk governance of the respective module for assessing consistency of meeting/maintaining product design or production requirements associated with the respective module.

6. The system of claim 1, wherein the QC records for a regulatory module and a post-market module of the plurality of modules comprise clinical or testing data and regulations for an approval or certification of the medical device under government regulations.

7. The system of claim 1, wherein the QC records for a local requirements module comprise clinical or testing data and regulations for an approval or certification of the medical device under a localized or regional government regulations.

8. The system of claim 1, wherein the QC records for a change control module comprise changes or proposed changes to the design, manufacture, and/or distribution of the medical device product, and tracking of the version information about the changes and applicability to particular regulatory rules or regulations.

9. The system of claim 1, wherein the QC records for a digital controls module comprise testing data and tracking of digital components of the medical device for compliance with regulations governing personal identifiable information and data security.

10. The system of claim 1, wherein the QC records for a service and installation module comprise data and tracking of a globally aligned service and installation process.

11. The system of claim 1, wherein the interface configured for managing the library of QC modules is configured to execute a process of generating modules in a plurality of waves, a first wave comprising modules associated with system management, training, and document control.

12. The system of claim 11, wherein the plurality of waves comprises a second wave of modules associated with auditing, validation, change controls, and purchasing controls and a third wave of modules associated with design and development, infrastructure, commercialization, regulatory requirements, and local requirements.

13. The system of claim 1, wherein the interface is configured to provide details of QC tasks arranged in fields of progressively increased levels of detail.

14. The system of claim 1, wherein the interface is configured to dynamically generate records and/or user notifications for new tasks based on particular timelines and/or a creation and/or partial completion of other QC tasks.

15. A computer implemented method for controlling the quality of a medical device or software product, the method comprising:
a) generating a quality control library of records for a quality control management system;
b) generating and configuring a plurality of quality control (QC) modules for managing a quality control process, wherein the configuring comprises:
c) assigning quality control tasks for each module to be performed for the quality control process;
d) assigning QC roles and responsibilities for the quality control tasks to a plurality of users of the QC modules;
e) assigning records of the library to the QC modules including information associated with the QC tasks and governmental rules or regulations applicable to the respective QC module and medical device or software product;
f) generating a user interface configured to manage the QC modules and library records, wherein the managing and implementing comprises adding or removing modules, assigning users to roles within the modules and responsibility for quality control tasks associated with the modules, and adding, removing, or modifying the quality control library records of a respective module.

16. The method of claim 15 wherein the plurality of quality control modules comprises two or more of:
a) a management responsibility module configured for QC tasks that assign users to management roles and responsibilities within a management system module;
b) a management system module configured for QC tasks that train users and assign tasks for management of a quality control process;
c) an improvement and resolution management module configured for QC tasks that track and improve the performance of the QC tasks of the management system module;
d) a post-market module configured for QC tasks that track issues associated with the medical device product after release of the product to users of the product;
e) an information documentation module configured for QC tasks that receive and store information associated with development and distribution of the medical device product; and
f) a training module configured for QC tasks that train users assigned to roles within the quality control modules.

17. The method of claim 15 wherein the plurality of quality control modules comprises two or more of:
a) an auditing module configured for QC tasks that audit quality control tasks and records of the plurality of modules;
b) a handling, storage, and distribution module configured for QC tasks that assess process(es) of handling, storing, and distribution of the medical product;
c) a qualification and validation module configured for QC tasks that qualify and validate the medical device product according to specification(s) and/or regulation(s);
d) a purchasing controls module configured for QC tasks that track and assess requisitions of components of the medical device;
e) a service and installation module configured for QC tasks that assess process(es) of servicing and installing the medical device for a user of the device;
f) a change controls module configured for QC tasks that track and assess changes in the designs, manufacture, installation, and/or servicing of the medical device;
g) a clinical evidence and studies module configured for QC tasks that track and assess the quality of clinical evidence and studies of testing or use of the medical device.

18. The method of claim 15 further comprising assigning and managing governance and implementation roles for users of the interface and library, wherein the governance roles are assigned privileged modular and/or inter-modular access and responsibility over respective module(s) and wherein implementation roles are assigned modular access and implementation responsibility of respective task(s) within a module.

19. The method of claim 15, further comprising assigning and managing enabler roles for one or more of the modules, wherein the enabler roles are assigned tasks for data analytics and risk governance of the respective module for assessing consistency of meeting/maintaining product design or production requirements associated with the respective module.

20. The method of claim 15 wherein the QC records for a regulatory module and a post-market module of the plurality of modules comprise clinical or testing data and regulations for an approval or certification of the medical device under government regulations.
